(19) United States
(12) Patent Application Publication
(10) Pub. No.: US 2006/0035848 A1
(43) Pub. Date: Feb. 16, 2006

(54) Broad-spectrum inhibitor of viruses in the Flaviviridae family

(75) Inventor: Joshua O. Ojwang, Edmond, OK (US)

Correspondence Address: FULBRIGHT & JAWORSKI L.L.P., 600 CONGRESS AVE., SUITE 2400, AUSTIN, TX, 78701, US

(73) Assignee: ZymeTx, Inc.

(21) Appl. No.: 11/199,795

(22) Filed: Aug. 9, 2005

Related U.S. Application Data
(60) Provisional application No. 60/599,954, filed on Aug. 9, 2004.

(51) Int. Cl. A61K 31/7076

(52) U.S. Cl. 514/43

(57) ABSTRACT

The present invention relates generally to the fields of chemistry and molecular biology. More particularly, it concerns the use of compounds to treat viral infection. In a preferred embodiment, 2-amino-8-(β-D-ribofuranosyl) imidazo [1,2-a]-s-triazine-4-one may be used to treat infection by viruses of the Flaviviridae family.

This application claims benefit of priority to U.S. Provisional Application Ser. No. 60/599,954, filed Aug. 9, 2004, the entire contents of which is hereby incorporated by reference in its entirety.

The government has rights in the present invention pursuant to grant number 1R43AI049592-01A1 from the National Institute of Allergy and Infectious Diseases.

### BACKGROUND OF THE INVENTION

1. Field of the Invention

The present invention relates generally to the fields of chemistry and molecular biology. More particularly, it concerns the use of compounds to treat viral infection including infection by viruses of the Flaviviridae family.

2. Description of Related Art

Flaviviridae is a family of more than 70 viruses, of which almost half have been associated with human disease. The most well-known are hepatitis C virus (HCV), dengue fever virus (DV), yellow fever virus (YFV), West Nile virus (WNV) and Japanese encephalitis virus (JEV). In addition, flaviviruses also cause disease in domestic or wild animals of economic importance.

HCV infection is the most common chronic blood-borne infection in the United States. There are about 36,000 new infections every year, of which 25-30% are symptomatic. It is estimated that 3.9 million (1.8%) Americans have been infected (Alter, 1995; Alter et al.; 1992, Barrera et al., 1995; NIH 1997; Prince et al., 1993; Thomas et al., 1995). The mosquito-borne flavivirus, dengue, is estimated to cause 100 million cases of dengue fever, 500,000 cases of dengue hemorrhagic fever and 25,000 deaths each year with 2.5 billion people at risk world wide (Monath, 1994). West Nile virus (WNV) is the causative agent of West Nile (WN) fever. The common complication is encephalitis (George et al., 1984). WN fever is a mosquito-born flavivirus infection that is transmitted to vertebrates primarily by various species of Culex mosquitoes. Like other members of this serogroup of flaviviruses, WNV is maintained in a natural cycle between arthropod vectors and birds. The first known human case of WNV infection recorded in the Western Hemisphere was reported in August 1999 (CDC, 1999); eventually, 62 cases of the disease were later confirmed (CDC, 2000). This outbreak was concurrent with increased mortalities among birds and horses. Initially, 70% of the human laboratory confirmed cases occurred within a 10-km radius, centered in the northern end of the New York City borough of Queens (CDC, 1999); however, recent reports have shown that this virus has persisted over the years in the United States. It has spread to other states on the eastern seaboard during 2000 and 2001, suggesting that WNV is now endemic in the United States and that its geographic range probably will continue to expand until it extends over much of the continent (CDC, 2001). In 2002 and 2003 the spread of WNV reached epidemic proportion (CDC). The viruses in the Flaviviridae family possess a single-stranded RNA genome of positive polarity. This genome expresses its proteins via translation of a single, long, open reading frame.

Although a successful vaccine against the prototypical flavivirus, yellow fever virus, has been in use since the 1930s, and vaccines to two other flaviviruses, Japanese encephalitis virus and tick-borne encephalitis virus, are currently available, at this time there are no vaccines approved for dengue fever, WN infection and HCV infection. Furthermore, ribavirin, which is used in combination with interferon (IFN) as the first-line therapy for many of the viruses in this family, adds an additional toxic side effect to the treatment (Markland et al., 2000). The side effect of ribavirin is anemia, which results from the accumulation of the triphosphate form of the drug in erythrocytes. As a result, there is a great need to develop new compounds to be used alone or in combination with IFN to improve efficacy and safety in patients infected with these viruses.

### SUMMARY OF THE INVENTION

The present invention provides a class of compounds, including 2-amino-8-(β-D-ribofuranosyl) imidazo [1,2-a]-s-triazine-4-one (ZX-2401), which possess broad-spectrum antiviral activity and are particularly effective against RNA-type viruses, including those belonging to the Flaviviridae family. In certain embodiments of the present invention, ZX-2401 may be used to treat infection by yellow fever virus (YFV), bovine viral diarrhea virus (BVDV), banzi virus (BV), dengue virus (DV), and/or West Nile Virus (WNV).

Another aspect of the present invention relates to a method for treating a Flaviviridae infection comprising administering to a subject a compound having the structure: 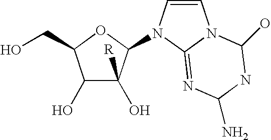
wherein R is chosen from the group consisting of hydrogen, halogen, alkyl, alkoxy, SH, and NH2. R may be chosen from the group consisting of F, Cl, I, Br, SH, NH₂, CH₃, and —OCH₃. R may be hydrogen. The Flaviviridae infection may comprise, in certain embodiments, a Hepacivirus infection, a Flavivirus infection, and/or a Pestivirus infection. The Hepacivirus infection may comprise infection with a hepatitis C virus. The Flavivirus infection may comprise infection with a yellow fever virus, a dengue virus, a tick-borne encephalitis virus, a St. Louis encephalitis virus, a Japanese encephalitis virus, a Murray Valley encephalitis virus, a Banzi virus, or a West Nile virus. The Pestivirus infection may comprise infection with a bovine viral diarrhea virus, a classical swine fever or hog cholera virus, or a border disease virus. The subject may be a mammal, and, in certain embodiments of the present invention, the mammal is a human, cow, dog, sheep, pig, cat, horse, mouse, or rat. The compound may be administered to the subject intranasally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation, injection, infusion, continuous infusion, localized perfusion bathing target cells directly, via a catheter, via a lavage, in cremes, in lipid compositions, or by any combination of the forgoing. The compound may be administered in a pharmaceutically acceptable carrier, diluent or vehicle. In certain embodiments the method may further comprise administering to the subject a second anti-viral composition. The second anti-viral composition may be interferon, ribavirin, ribavirin-2′,3′,5′-triacetate, polyriboinosinic-polyribocytidylic acid, 10-carboxymethyl-9-acridanone, mycophenolic acid, EICAR, tiazofurin, selenazofurin, a polyanion, a bicyclam, pirodavir, polysulfate PAVAS, or a plant lechtin. The interferon may be α-2b interferon, α-2a interferon, consensus interferon, or α-1n interferon.

Another aspect of the present invention relates to a compound having the structure: 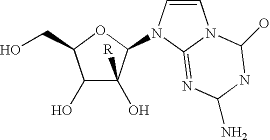
wherein R is chosen from the group consisting halogen, alkyl, alkoxy, SH, and NH₂. In certain embodiments of the present invention, R is F, Cl, I, Br, SH, NH₂, CH₃, or —OCH₃.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve the methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B: FIG. 1A, Evaluation of ZX-2401 against HCV was conducted using HCV Replicon assay. The experiment was carried out at Apath LLC (St. Louis, Mo.) according the Apath HCV replicon assay protocol. ZX-2401 displayed a dose-responsive anti-HCV replicon effect. FIG. 1B, The cytotoxicity of ZX-2401 was also determined by measuring the effect on GAPDH mRNA. ZX-2401 displayed no toxicity as measured by GAPDH mRNA levels.

### DESCRIPTION OF ILLUSTRATIVE EMBODIMENTS

The inventor has identified a class of compounds, including 2-amino-8-(β-D-ribofuranosyl) imidazo [1,2-a]-s-triazine-4-one (ZX-2401), which possess broad-spectrum antiviral activity and are particularly effective against RNA-type viruses, including those belonging to the Flaviviridae family. In certain embodiments of the present invention, ZX-2401 may be used to treat infection by yellow fever virus (YFV), bovine viral diarrhea virus (BVDV), banzi virus (BV), dengue virus (DV), and/or West Nile Virus (WNV).

The compound 2-amino-8-(β-D-ribofuranosyl) imidazo [1,2-a]-s-triazine-4-one (ZX-2401) was originally synthesized and tested against picomaviruses (Kim et al, 1978). This investigation showed that the compound was markedly active against vesicular stomatitis, coxsackie B-1 virus and Echo-6 virus, and moderately active against five rhinoviruses (Kim et al., 1978). U.S. Pat. No. 4,246,408 and WO 01/17518 describe the use of ZX-2401 to treat infection by viruses such as the influenza virus. However, no evidence has supported the use of this compound for the treatment of infection by viruses of the Flaviviridae family.

I. ZX-2401 and Derivatives

ZX-2401 (2-amino-8-(β-D-ribofuranosyl) imidazo [1,2-a]-s-triazine-4-one, 5-aza-7-deazaguanosine) and ZX-2401 derivatives are presented in the present invention for use in treating viral infections, preferably Flaviviridae infections. Derivatives of ZX-2401 include compounds with the structure: 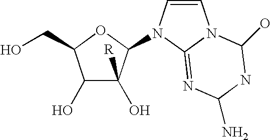
wherein X is chosen from the group consisting of halogen, alkyl, alkoxy, alkenyl, alkynyl, SH, and NH₂. R is preferrably chosen from the group consisting of F, Cl, I, Br, SH, NH₂, CH₃, and —OCH₃. When R is hydrogen, the compound is ZX-2401.

An “alkyl” group refers to a saturated aliphatic hydrocarbon, including straight-chain, branched chain, and cyclic alkyl groups. Alkyl groups can comprise any combination of acyclic and cyclic subunits. Further, the term “alkyl” as used herein expressly includes saturated groups as well as unsaturated groups. Unsaturated groups contain one or more (e.g., one, two, or three), double bonds and/or triple bonds. The term “alkyl” includes substituted and unsubstituted alkyl groups. When substituted, the substituted group(s) may be hydroxyl, cyano, alkoxy, ═O, ═S, NO₂, N(CH₃)₂, amino, or SH. Preferably, the alkyl group has 1 to 12 carbons. More preferably, it is a lower alkyl of from 1 to 7 carbons.

An “alkenyl” group refers to an unsaturated hydrocarbon group containing at least one carbon-carbon double bond, including straight-chain, branched-chain, and cyclic groups. Preferably, the alkenyl group has 1 to 12 carbons. More perferably it is a lower alkenyl of from 1 to 7 carbons. The alkenyl group may be substituted or unsubstituted. When substituted, the substituted group(s) is preferably hydroxyl, cyano, alkoxy, ═O, ═S, NO2, N(CH₃)₂, halogen, amino, or SH.

An “alkynyl” group refers to an unsaturated hydrocarbon group containing at least one carbon-carbon triple bond, including straight-chain, branched chain, and cyclic groups. Preferably, the alkynyl group has 1 to 12 carbons. More perferably it is a lower alkynyl of from 1 to 7 carbons. The alkynyl group may be substituted or unsubstituted. When substituted, the substituted group(s) is preferably hydroxyl, cyano, alkoxy, ═O, ═S, NO2, N(CH₃)₂, amino, or SH.

An “alkoxy” group refers to an “—O-alkyl” group, where “alkyl” is defined above.

II. Viral Diseases

As presented in the present invention, ZX-2401, and derivatives thereof, may be used to treat viral diseases. Viral diseases include, but are not limited to influenza A, B and C, parainfluenza (including types 1, 2, 3, and 4), paramyxoviruses, Newcastle disease virus, measles, mumps, adenoviruses, adenoassociated viruses, parvoviruses, Epstein-Barr virus, rhinoviruses, coxsackieviruses, echoviruses, reoviruses, rhabdoviruses, lymphocytic choriomeningitis, coronavirus, polioviruses, herpes simplex, human immunodeficiency viruses, cytomegaloviruses, papillomaviruses, virus B, varicella-zoster, poxviruses, rubella, rabies, picomaviruses, rotavirus, Kaposi associated herpes virus, herpes viruses type 1 and 2, hepatitis (including types A, B, and C), and respiratory syncytial virus (including types A and B).

A. Flaviviridae

The Flaviviridae family of viruses includes Hepaciviruses (i.e., Hepacivirus), Flaviviruses (i.e., Flavivirus), and Pestiviruses (i.e., Pestivirus). Flaviviridae are RNA viruses that possess a single-stranded RNA genome of positive polarity. Although viruses belonging to these different genera (i.e., Hepacivirus, Flavivirus, and Pestivirus) generally have different biological properties and typically do not show serological cross-reactivity, significant similarity in terms of virion morphology, genome organization, and presumed replication strategy have been observed (Chambers et al., 1990; Rice et al., 1996; Westaway, 1987).

Infection by Flaviviridae presents a serious problem for both humans and non-human animals including livestock. Infection by Flaviviridae may result in adverse symptoms including encephalitis and/or death. Flaviviridae that may infect humans include West Nile virus (WNV), yellow fever virus (YFV), Japanese encephalitis virus (JEV), Kyasanur Forest virus, Murray Valley encephalitis virus, Omsk hemorrhagic fever virus, Rocio virus, Central European encephalitis virus, Russian spring-summer encephalitis virus, Hepatitis C virus (HCV), Dengue virus (types 1, 2, 3 and 4), tick borne encephalitis virus, and St. Louis encephalitis virus. The compounds of the present invention, preferably ZX-2401 and derivatives thereof, may be used to treat Flaviviridae infections in human and non-human animals.

Hepatitis G virus/GB-virus C is classified within the family Flaviviridae but presently has not been assigned to a genus. GB viruses (GBV-A, GBV-B, and GBV-C) are phylogenetically related to the hepatitis C virus (Karayiannis et al., 1998; Linnen et al., 1996; Yamada et al., 1998). GBV-A and GBV-B infection typically occurs in tamarins; humans are typically the host for GBV-C. GBV-C is often found as a co-infection associated with the hepatitis C virus and is transmitted in the same way.

1. Flaviviruses

Currently, more than 70 flaviviruses have been reported, and many of them cause important human diseases. All human flaviviruses can be transmitted by vectors such as ticks and mosquitoes; thus these diseases are very difficult to eradicate (Monath et al., 1996). Based on phylogenetic analysis, 72 species of flaviviruses have been grouped into 14 clades, which in turn can be grouped in three clusters: the mosquito-borne cluster, the tick-borne cluster, and the no-vector cluster. The flaviviruses that infect humans typically belong to the first two clusters. The last cluster includes a few viruses which have been isolated from mice or bats; however, no arthropod vector or natural route of transmission has yet been demonstrated (Kuno et al., 1998).

Flaviviruses include yellow fever virus (YFV), dengue virus (DV), Japanese encephalitis virus (JEV), Russian spring-summer encephalitis virus, Murray Valley encephalitis virus, St. Louis encephalitis virus, Omsk hemorrhagic fever virus, Kyasanur forest disease virus, Louping ill virus, and West Nile virus (WNV). In a preferred embodiment of the present invention, ZX-2401 and/or ZX-2401 derivatives may be used to treat infection by flaviviruses.

Flaviviruses present significant problems for human and non-human animals. Although a vaccine exists against YFV, this virus is still a leading cause of hemorrhagic fever and related mortality (up to 50%) worldwide (Monath, 1987). Over 100 million cases of DV and at least 500,000 cases of dengue hemorrhagic fever (DHF), including in about 25,000 fatal cases, have been reported to occur annually in humans worldwide (Gubler, 1998; Rice, 1996; World Health Organization, 1997). The isolated DV strains have been isolated can divided into four serotypes (types 1, 2, 3, and 4) (Gubler, 1998; World Health Organization, 1993). Although the primary infection by DV is often subclinical, particularly in children, and appears to induce a lifelong immunity for that particular serotype, a second infection with a different serotype may lead to the development of DHF or dengue shock syndrome (combined mortality, up to 5%) (Kurane et al., 1994; Ramirez Ronda et al., 1994). DHF has been classified into four grades according to the severity of shock and bleeding (World Health Organization, 1993). International travel and uncontrolled urbanization have resulted in an increased spread of the mosquito vector (Aedes aegypti and Aedes albopictus). DV infections in most tropical and subtropical regions now are hyperendemic (prevalence of two or more DV serotypes), which enhances the occurrence of DHF and dengue shock syndrome (Gubler, 1998). JEV is transmitted by mosquitoes and is the leading cause of viral encephalitis worldwide. Approximately 50,000 cases occur annually in Asia and result in high mortality (30%) or in long-lasting neurological sequelae (30%) (Kalita and Misra, 1998; Misra et al., 1998).

Other important flaviviruses that cause encephalitis are also responsible for high mortality rates and/or neurological sequelae. For example, two important subtypes of tick borne encephalitis virus exist, i.e., the European and Eastern subtypes. The mortality rate associated with infection by the Eastern subtype (also referred to as Russian spring-summer encephalitis virus) has been reported to be ˜20%; for infection by the Western subtype (also referred to as Central European encephalitis virus) this value has been estimated to be 1 to 2% (Heinz and Mandl, 1993). Although the last large epidemic caused by Murray Valley encephalitis virus (MVEV) occurred in 1974, new cases of MVEV infection have been reported regularly, particularly in Western Australia (Mackenzie and Broom, 1995). West Nile virus (WNV) is endemic in Africa, the Middle East, and near the Mediterranean Sea. However, in 1996 an outbreak of WNV infection with 373 cases and 17 deaths was reported in Romania (Han et al., 1999; Tsai et al., 1998), and more recently, in 1999 in the New York City area, an outbreak of WNV resulted in an estimated 77 cases of infection including six deaths. Although there are no recent reports of outbreaks or epidemics of St. Louis encephalitis virus, the virus causing this disease is endemic in the western United States (Kramer et al., 1997). Omsk hemorrhagic fever virus is responsible for a number of infections annually in rural areas in the Omsk region in Russia. Annually, 400 to 500 virologically diagnosed cases of Kyasanur forest disease virus infections are reported in India (Monath and Heinz, 1996). Louping ill virus (LIV) primarily infects sheep, although this virus has the potential to infect humans (Davidson et al., 1991).

2. Hepaciviruses

Hepaciviruses include the hepatitis C virus (HCV). It is estimated that approximately 3% of the world's population (170 million people) are infected with HCV (Lavanchy et al., 1999). Infection by HCV increases risk of developing cirrhosis and/or liver cancer.

It is contemplated that ZX-2401, and derivatives thereof, may be used to treat infection by Hepaciviruses, preferably infection by hepatitis C. Currently, chronic or early-diagnosed acute hepatitis C is typically treated with α-2 interferon alone (Lau et al., 1998) or in combination with ribavirin (Davis, 1999; Davis et al., 1998). The interferons approved for HCV are α-2b interferon (Intron-A), α-2a interferon (Roferon-A), consensus interferon (r-metIFN-Con1), and α-1n interferon (Welferon). Interferon therapy, which is expensive, is associated with many side effects, particularly after prolonged therapy, and is effective in only a subset of patients (Martinot Peignoux et al., 1998). It has been estimated that approximately forty percent of patients with chronic HCV infection have an initial response to interferon therapy but may subsequently relapse. The limited approaches to and deleterious side-effects associated with current therapies for HCV infection emphasize the need for new therapies.

3. Pestiviruses

Pestiviruses typically infect non-human animals, and pestiviruses include bovine viral diarrhea virus (BVDV), classical swine fever virus, and border disease virus. Although these viruses typically do not infect humans, pestiviruses have been shown to be able to cross the interspecies barrier (Edwards et al., 1995; Terpstra and Wensvoort, 1997; Van Campen et al., 1997). Thus it is possible that these viruses may in the future infect humans.

BVDV infections are associated with severe mucosal disease in cattle, although swine and other ruminants are also susceptible to the virus (Meehan et al. 1998; Terpstra and Wensvoort, 1998). BVDV often results in the death of an infected cow or bull. Classical swine fever virus, also known as hog cholera virus, is an important, highly contagious pathogen of swine that is easily transmitted by aerosol, contaminated clothing, or direct contact (Laevens et al., 1998a; Laevens et al., 1998b). Infection of a pig by classical swine fever virus typically results in death. Border disease virus can infect sheep and goats. Pestiviruses result in significant economic losses. Although non-human animals infected by pestiviruses are typically slaughtered in an attempt to prevent the spread of the disease, in some instances it may be preferable to treat infected animals. Additionally, if a human became infected with a pestivirus, then this would warrant treatment. In an embodiment of the present invention, ZX-2401 and ZX-2401 derivatives may be used to treat infection by pestiviruses.

III. Pharmaceutical Preparations

Pharmaceutical compositions of the present invention comprise an effective amount of one or more ZX-2401, or a ZX-2401 derivative, or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one ZX-2401, or a ZX-2401 derivative, or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The ZX-2401, or a derivative thereof, may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The ZX-2401 or ZX-2401 may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include ZX-2401 or a ZX-2401 derivative, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the ZX-2401, or ZX-2401 derivative, may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present invention, the ZX-2401, or a derivative thereof, are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft- shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et aL., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically- effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, ZX-2401, or a derivative thereof, may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,753,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound ZX-2401 or the ZX-2401 derivative may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725, 871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

IV. Combination Therapy

In order to increase the effectiveness of ZX-2401 or a ZX-2401 derivative, it may be desirable to combine these compositions and methods of the invention with an agent effective in the treatment of infection by a virus, preferably a Flaviviridae. In some embodiments, it is contemplated that a conventional therapy or agent, including but not limited to, a pharmacological therapeutic agent, a surgical therapeutic agent (e.g., a surgical procedure) or a combination thereof, may be combined with ZX-2401 and/or ZX-2401 derivative administration. In a non-limiting example, a therapeutic benefit comprises reduced viral titer in a subject and/or reduced symptoms of viral infection. Thus, in certain embodiment, a therapeutic method of the present invention may comprise administration of a ZX-2401 and/or a ZX-2401 derivative of the present invention in combination with another therapeutic agent.

This process may involve contacting the cell(s) with an agent(s) and the ZX-2401 and/or ZX-2401 derivative at the same time or within a period of time wherein separate administration of the ZX-2401 and/or ZX-2401 derivative and an agent to a cell, tissue or organism produces a desired therapeutic benefit. The terms “contacted” and “exposed,” when applied to a cell, tissue or organism, are used herein to describe the process by which a therapeutic construct of the present invention and/or therapeutic agent are delivered to a target cell, tissue or organism or are placed in direct juxtaposition with the target cell, tissue or organism. The cell, tissue or organism may be contacted (e.g., by adminstration) with a single composition or pharmacological formulation that includes both a ZX-2401, and/or a ZX-2401 derivative, and one or more agents, or by contacting the cell with two or more distinct compositions or formulations, wherein one composition includes a ZX-2401 and/or a ZX-2401 derivative and the other includes one or more agents.

The ZX-2401 or ZX-2401 derivative may precede, be co-current with and/or follow the other agent(s) by intervals ranging from minutes to weeks. In embodiments where the ZX-2401 and/or ZX-2401 derivative and other agent(s) are applied separately to a cell, tissue or organism, one would generally ensure that a significant period of time did not expire between the time of each delivery, such that the ZX-2401 and/or ZX-2401 derivative and agent(s) would still be able to exert an advantageously combined effect on the cell, tissue or organism. For example, in such instances, it is contemplated that one may contact the cell, tissue or organism with two, three, four or more modalities substantially simultaneously (i.e. within less than about a minute) as the treatment for a viral infection. In other aspects, one or more agents may be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 1 week, about 2 weeks, about 3 weeks, about 4 weeks, about 5 weeks, about 6 weeks, about 7 weeks, about 8 weeks, about 1 month, about 2 months, about 3 months, about 4 months, about 5 months, about 6 months, about 7 months, about 8 months, about 9 months, about 10 months, about 11 months, or about 12 months, and any range derivable therein, prior to and/or after administering the ZX-2401 and/or ZX-2401 derivative.

Various combination regimens of the ZX-2401 or ZX-2401 derivative and one or more agents may be employed. Non-limiting examples of such combinations are shown below, wherein a composition ZX-2401 or ZX-2401 derivative is “A” and an agent is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the composition ZX-2401 or ZX-2401 derivative to a cell, tissue or organism may follow general protocols for the administration of vascular or cardiovascular therapeutics, taking into account the toxicity, if any. It is expected that the treatment cycles would be repeated as necessary. In particular embodiments, it is contemplated that various additional agents may be applied in any combination with the present invention.

Agents that may be used in combination with the present invention include anti-viral compounds; for example, compounds that inhibit viral entry, inhibit IRES, inhibit protease, inhibit RNA-dependent RNA polymerase, and /or inhibit helicase may be used in combination with the present invention. Additionally, ribozyme therapy, gene therapies, and/or antisense oligionucelotide therapies may also be used in combination with the present invention. Preferred agents that may be used in combination with the present invention include ribavirin, ribavirin-2′,3′,5′-triacetate, polyriboinosinic-polyribocytidylic acid, 10-carboxymethyl-9-acridanone, mycophenolic acid, EICAR, tiazofurin, selenazofurin, a polyanion, a bicyclam, pirodavir, polysulfate PAVAS, and plant lechtins. Interferons (e.g., α-2b interferon, α-2a interferon, consensus interferon, and/or α-1n interferon) may be used in combination with ZX-2401 or a ZX-2401 derivative.

A. Compounds that inhibit Viral Entry

Molecules that specifically interfere with the initial steps (binding and penetration) of viral interaction with the host cell may be used in combination with compounds of the present invention. These compounds may interfere with the viral molecules that bind to the host cell.

The polysulfate PAVAS (a copolymer of acrylic acid and vinyl alcohol sulfate) may be used in combination with compounds of the present invention. Polysulfate PAVAS has been shown to block the infection of primary hepatocytes with HCV (Clarysse et al., 1998). Other polyanionic (sulfate, sulfonate, carboxylate, oxometalate, etc.) substances and derivatives of polysulfate PAVAS may also be used in combination with the present invention.

Other compounds that may be used with the present invention include: polyanions (such as sulfated, sulfonated, or carboxylated polymers and polyoxymetalates that inhibit the binding to the host cell of enveloped viruses, plant lectins (e.g., either mannose, N-galactosamine, or N-acetylglucosamine specific) that display anti-viral properties, pirodavir, and bicyclams (e.g., AM3100).

B. Compounds that Inhibit IRES

Compunds that inhibit IRES may also be used in combination with the present invention. An example of a possible strategy to discover inhibitors of IRES has been described (Cai et al., 1998). Compounds such as siRNA that inhibit viral replication (e.g., by competing for critical cellular polypeptides that are required for viral IRES-mediated translation (Lu and Wimmer, 1996; Zhao et al., 1999)) may also be used with the present invention.

C. Compounds that Inhibit Capping

In Flaviviridae, prevention of capping may elicit an antiviral effect by “disabling” the RNA of the progeny virus. Caps are synthesized by an RNA triphosphatase, a guanylyltransferase, and a methyltransferase. Viral (and cellular) methyltransferases are sensitive to inhibition by S-adenosylhomocysteine (SAH). Compounds that inhibit the viral enzymes (i.e., RNA triphosphatase and methyl transferase) involved in capping, as well as the cellular SAH hydrolase, may inhibit flaviviruses and be used in combination with compounds of the present invention. For example, the SAH hydrolase inhibitor 3-deazaneplanocin A may be used in combination with ZX-2401 and/or ZX-2401 derivatives.

D. Compounds that Inhibit Protease

Compounds that inhibit the Flaviviridae proteases may be used with the present invention. For example compounds that inhibit the NS2/3 (putative metalloproteinase) and the NS3 (serine protease) of HCV may be used in combination with compounds of the present invention. The HCV serine protease is encoded by the NS3 gene and may be an important target for antiviral therapy for members of the Flaviviridae. Several methods methods and approaches exist to discover protease inhibitors. For example, Sudo et al. (1996) engineered a maltose-binding protein-NS3-NS4A fusion protein and a synthetic peptide that mimics the NS5A-NS5B junction, which is cleaved by the NS3 protease. Nonpeptidic (Kakiuchi et al., 1998; Sudo et al., 1997) or peptidic (Ingallinella et al., 1998; Steinkuhler et al., 1998) inhibitors of the HCV protease have been identified and may be used in combination with compounds of the present invention.

E. Compounds that Inhibit the RNA-Dependent RNA Polymerase

Compounds that inhibit the RNA-dependent RNA polymerase (RdRp) may also be used in combination with compounds of the present invention. The RNA polymerases of HCV (Al et al., 1998; Lohmann et al., 1997; Yamashita et al., 1998, Yuan et al., 1997; Behrens et al., 1996), DENV (Tan et al., 1996), and BVDV (Zhong et al., 1998) have been cloned and expressed. For example, the 5′-triphosphate metabolite of ribavirin is believed to act as an inhibitor of the viral RdRp (Huggins, 1989). In a preferred embodiment of the present invention, ribavirin or a ribavirin derivative (e.g., EICAR; De Clercq et al., 1991) may be used in combination with ZX-2401 or a ZX-2401 derivative.

F. Compounds that Inhibit Helicase

As well as the serine protease activity located at the N terminus of the NS3 protein, helicase and NTPase activities are located in the C terminus of this protein. Helicases are enzymes which unwind double-stranded -DNA, RNA-DNA, or RNA-RNA regions in an ATP-dependent reaction. The function of the helicase of the Flaviviridae is presumed to be the unwinding of the plus and minus RNA strands of the genome after the polymerase reaction. The helicase of the Flaviviridae belongs to the DEAD (Asp-Glu-Ala-Asp) box family of RNA helicases (Kim and Caron, 1998).

G. Ribozymes and Gene Therapy

Ribozyme therapies and/or gene therapies may be used in combination with compounds of the present invention. Ribozymes are RNA molecules composed of a catalytic site that can cleave a target RNA at a specific site and a sequence complementary to a designated site on the target RNA. Ribozymes targeted to highly conserved regions of the HCV genome were shown to cleave the viral RNA and to reduce in vitro translation (Ohkawa et al., 1997). The ribozymes reduced or eliminated HCV RNA expressed in cultured cells and in primary human hepatocytes that had been isolated from patients with advanced HCV-associated liver disease (Lieber et al., 1996). Due to the RNA structure of ribozymes and hence their high biodegradability, a significant delivery problem exists. A gene therapy approach, using for example adenovirus-mediated expression of ribozymes, may help to solve this problem (Lieber and Kay, 1996; Welch et al., 1996).

H. Antisense Oligonucleotide Therapy

Antisense oligonucleotides are able to target and disable viral replication by interfering with the translation process by hybrid arrest of the translational machinery or by the induction of RNase that results in the cleavage of the double-stranded RNA portion of the hybrid. Antisense (phosphorothioate) oligonucleotides have been shown to inhibit HCV translation in an in vitro model (Alt et al., 1997; Wakita et al., 1999). In certain embodiments, antisense oligonucleotide therapy may be used in combination with ZX-2401 and/or a ZX-2401 derivative.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Preparation of ZX-2401. The preparation was accomplished by the synthesis scheme previously reported by Kim et al. (1978), starting from commercially available cyanuric chloride. Briefly, selective amination of cyanuric chloride with gaseous ammonia at 0° C. followed by careful hydrolysis of one of the halogens gave 2-amino-4-chloro-6-hydroxy-1,3,5-triazine. Reaction of 2-amino-4-chloro-6-hydroxy-1,3,5-triazine with aminoacetaldehyde dimethyl acetal in aqueous basic media at reflux temperature furnished the intermediate. The acetals groups were hydrolyzed using 6N hydrochloric acid followed by ring annulation in concentrated sulfuric acid at 95° C. and gave crystalline 2-aminoimidazo[1,2-a]-s-triazine-4-one (5-aza-7-deazaguanine). Glycosylation was achieved by first converting this compound to its trimethylsilyl derivative in HMDS followed by treatment of this intermediate with 1-O-acetyl-2,3,5-tri-O-benzoyl-β-D-ribofuranose in anhydrous dichloroethane in presence of stannic chloride. The reaction was stereoselective and on purification gave only the β anomer. The protecting groups were removed in sodium methoxide in methanol and on recrystallization of the resulting crude product gave a good yield of 2-amino-8-(β-D-ribofuranosyl) imidazo [1,2-a]-s-triazine-4-one (ZX-2401, 5-aza-7-deazaguanosine).

The compound is a white powder, which was dissolved in water at 10 mg/kg to make a stock solution. The working solutions were prepared by diluting the stock solution in culture medium to appropriate concentrations needed for each assay. Ribavirin and IFN alpha B/D were from Ribopharm (a division of ICN Pharmaceuticals, Costa Mesa, Calif.). Units of IFN that were used were based upon the titer provided by Ribopharm for the full strength material (in international units/ml). A unit of IFN is defined as the amount causing a 50% reduction in the CPE of vesicular stomatitis virus in L929 cell culture.

Viruses. Banzi virus (BV), H 336 strain, was purchased from the America Type Culture Collection (ATCC), Manassas, Va. It was isolated from serum of a febrile boy in South Africa. Pools of the virus were prepared in Africa green monkey (Vero) cells. Vero cells were used for antiviral and cytotoxicity testing.

Dengue virus (DV) type 2, New Guinea strain, was obtained from the Centers for Disease Control and Prevention (CDC), Fort Collins; Colo. Pools of the virus were prepared in Vero cells. MA-104 cells (another African green monkey kidney cell line) were used for antiviral testing.

Bovine viral diarrhea virus (BVDV), TN131 strain, was obtained from Blair Fujimoto of Hyclone Laboratories, Logan, Utah, who obtained the virus from John Black, American BioResearch, Milton, Tenn. It was originally obtained from the spleen of a calf with diarrhea. Pools of the virus were made up in bovine turbinate (BT) cells. BT cells were used for antiviral testing.

Yellow fever virus (YFV), 17D strain, was obtained from ATCC. It was originally prepared from infected mouse brain. Pools of the virus were prepared in Vero cells. Vero cells were used for antiviral testing.

Two strains of WNV used were strain B956 (ATCC VR-82; ATCC, Manassas, Va.) and a New York isolate from homogenized crow brain (NY, CDC 996625, V1 D3 Nov. 10, 1999, Robert Lanciotti, CDC, Division of Vector-Borne Infectious Diseases, Ft. Collins, Colo.).

Cells and Media. The following cells and media were used with the appropriate virus: BT cells were obtained from ATCC. Growth medium was Eagle's minimum essential medium with non-essential amino acids (MEM), 10% fetal bovine serum (FBS) and 0.1% NaHCO₃ and 50 μg gentamicin/ml.

MA-104 cells were obtained from Whittaker MA Bioproducts, Walkersville, Md. Growth medium was MEM 199, 5%FBS, 0.1% NaHCO₃ without antibiotics. Test medium for DV was MEM, 2% FBS, 0.18% NaHCO₃ and 50 μg gentamicin/ml.

Vero cells were obtained from ATCC. Growth medium was MEM 199, 5% FBS, 0.1% NaHCO₃ without antibiotics. Test medium for BV and YFV was MEM, 2% FBS, 0.18% NaHCO₃ and 50 μg gentamicin/ml.

For WNV, African green monkey kidney cells (Vero 76, ATCC CCCL1587) were used. MEM with 1% FBS, 0.1% NaHCO₃, and 50 μg/mL gentamicin (Sigma, St. Louis, Mo.) were used to maintain cells during antiviral experiments. Virus stocks were prepared in MA104 cells and stored at −80° C. The viruses were titered in lightly confluent Vero cells in 96-well microtiter plates.

Assay Systems. The assay systems used in the experiments discussed in this report are described below.

Antiviral testing using CPE Assay. The CPE assay was performed as previously described by Smee et al. (1988). Briefly, the compounds were diluted in the same type of medium used to prepare the compounds, and appropriate cell types for each target virus were incubated overnight at 37° C. in order to establish a cell monolayer. When the monolayer was established, the growth medium was decanted, and various dilutions of test compound were added to each well (6 wells/dilution, 0.1 ml/well). Compound diluent medium was added to cell control wells and virus only control wells (0.1 ml/well). Virus, diluted in test medium, was then added to appropriate wells at 0.1 ml/well approximately 5 minutes after the compound. Test medium without virus was added to all toxicity control wells (2 wells/dilution of each test compound) and to cell control wells at 0.1 ml/well. The plates were sealed with plastic wrap (Saran®) and incubated at 37° C. in a humidified incubator with 5% CO₂, 95% air atmosphere, until the virus control wells had adequate cytopathic effect (CPE) readings. This was usually achieved after 72 hr. Cells were then examined microscopically for virus-induced CPE, which was scored from 0 (normal cells) to 4 (maximal, 100%, CPE). The cells in the toxicity control wells were observed microscopically for morphologic changes attributed to cytotoxicity at the same time. The cytotoxicity was also graded at T (100% toxicity, complete cell sloughing from plate), PVH (80% cytotoxicity), PH (60% cytotoxicity), P (40% cytotoxicity), PS (20% cytotoxicity), and 0 (normal cells.). The 50% effective concentration (EC₅₀) and 50% cytotoxic concentration (CC₅₀) were calculated by regression analysis of the virus CPE data and the toxicity control data, respectively. The selectivity index (SI) for each test compound was calculated by the formula SI=CC₅₀÷EC₅₀.

Neutral Red Testing Procedures. The neutral red evaluation was conducted as previously described by Player et al. (1998). Briefly, the plates were first read visually for cytopathology and toxicity, after which 0.1 ml of sterile neutral red (0.034% physiological saline solution) was added to each well. The plates were wrapped in aluminum foil to eliminate light exposure and placed at 37° C. for 1-2 hours. All medium was removed, and the cells were washed twice (0.2 ml/well for each wash) with phosphate buffered saline. The plates were inverted and allowed to drain on a paper towel. Neutral red was extracted from the cells by adding 0.2 ml of an equal volume mixture of absolute ethanol and Sorensen citrate buffer, pH 4, to each well and placing the plates at 540 nm with a Model EL309 microplate reader (Bio-Tek Instruments, Inc., Winooski, Vt.). The EC₅₀ and CC₅₀ were calculated by regression analysis. The SI for each compound tested was calculated using the formula: SI=CC₅₀÷EC₅₀.

Virus yield reduction (VYR) assay. To delineate the actual antiviral effects of test compounds from the potential cytotoxic effects of the compounds, the infectious virus recovered from the antiviral assays was quantified using the VYR assay (Smee et al., 1992) for various days of post-infection cultures. The test method as described above for visual inhibition of CPE assay was used; inhibition of CPE was read visually. The 96-well plate was then frozen at minus 80° C. and thawed, and the virus from the supernatants was then assayed by using a series of 10-fold dilutions and assaying each in quadruplicate in a monolayer of Vero cells grown in 96-well microplates. Viral CPE was determined visually 6 days later after incubation at 37° C. The virus titer in relation to the concentration of test compound was plotted to determine a 90% effective concentration (EC₉₀), the dose that reduced virus titer by 1 log₁₀.

HCV Replicon Assay System. HCV replicon-containing cells were plated onto the wells of a 96-well plate at 12×10³ cells/well and allowed to adhere for 3 hours. Compounds were diluted as specified in complete media before addition to cell monolayers. Human IFN α (200 IU/ml) was used as a positive control for decrease of cellular replicon levels. Untreated cells were used as a negative control. After cells were treated for 24 hours, total RNA was extracted using the Qiagen 96-well RNeasy kit. All compound concentrations tested, as well as controls, were done in quadruplicate. Replicon RNA was measured using “real-time” quantitative RT-PCR and primers specific for the 3′ NTR of HCV. Amplicon was detected and quantified using syber green fluorescence detection. Data were expressed as threshold cycle number and plotted as a percent of untreated control. The larger the amount of target RNA template present, the fewer cycles were required to reach threshold. Duplicate serial 1:3 dilutions of untreated Ava.5 total RNA were done in the following manner: 1:3, 1:9 and 1:27, and an average taken and plotted on a linear regression curve. All threshold cycle data from the compounds tested were plotted against this curve to obtain percent of untreated control. Nonspecific cellular effects of compounds were assessed by measuring glyceraldehyde-3-phosphate dehydrogenase (GADPH) mRNA using quantitative RT-PCR with primers specific for GAPDH mRNA.

Combination experiments. From previous experiments conducted to evaluate YFV, it was recognized by the inventor that the virus may be present inside of cells even though virus-induced CPE may be completely reduced. For this reason, the CPE assay alone is insufficient to get good quantitative data for a drug combination study. Thus, the virus yield reduction assay was also employed. This made the experiment a two-part study. In the first part, ZX-2401 and IFN were evaluated for inhibition of viral CPE alone or in combination. Before the start of the combination experiment, both ZX-2401 and the IFN were pre-titrated on cells to find doses reducing viral CPE. ZX-2401 was then used at 320, 100, 32 and 0 μg/ml. IFN was used at 100, 32, 10, 3.2 and 0 units/ml. All possible combinations, in a checkerboard fashion, were made for inhibition of the virus. These combinations were performed in 96-well plates, in a manner similar to that described above. After CPE was determined visually, the plate was frozen at −80° C. until the next day, then thawed. The cell/supernate from three infected wells from each dilution were pooled for virus titer determinations. Each sample was serially diluted in 10-fold increments on new confluent monolayers in 96-well plates, using 4 wells per dilution. End points were determined by the method of Reed and Muench (1938). Virus titers in each sample were expressed as 50% cell culture infectious doses (CCID50) per 0.1 ml. The statistical evaluation was performed on the data using the combination index method described by Schninazi et al. (1982).

### Example 2

### ZX-2401 Inhibits Viruses of the Flaviviridae Family

The viruses in the Flaviviridae family have recently received attention because of the increased incidences of HCV infection, isolation of WNV in North America, and lack of vaccines and cost effective therapies. The isolation of WNV in the Northern Hemisphere in particular has brought awareness that the viruses in this family are not confined to the tropics, and as such, proactive steps are needed to discover and develop therapeutic agents against these viruses. The lack of reliable in vitro culture systems and limited animal models have hampered attempts to identify and develop potential antiviral agents, especially for HCV.

Recently, a HCV replicon cell culture assay system (Lohmann et al., 1999) has been developed. This assay system uses HCV subgenomic constructs permanently or transiently transfected in HuH-7 human hepatoma cells. The availability of this HCV replicon system has allowed the investigation of anti-HCV compounds based on their ability to inhibit subgenomic HCV RNA replication in cell culture. This assay has some limitations (Ning et al., 1998; Crotty et al., 2001). This assay system is most logical for compounds that inhibit HCV RNA polymerase. Thus, the replicon system is not suitable for compounds that exert their effect prior to viral replication or later in the viral life cycle. For example, this assay might not be suitable for ribavirin, which has been reported to prevent HCV infection by either modulating the Th1:Th2 ratio (Ning et al., 1998) or induction of lethal mutagenesis after incorporation during viral RNA synthesis, which leads to loss in total viral genomic RNA (Crotty et al., 2001). Based on this observation, it is very important to investigate the anti-HCV activity of new compounds using all the available assay systems. One approach to the identification of potential inhibitors of these emerging microorganisms is the use of similar or related viruses. The most logical approach is, therefore, to identify a number of broad-spectrum compounds which are inhibitory to a number of these viruses in the hope that when an outbreak occurs, such as that of WNV in New York, drugs are available for immediate treatment.

Antiviral Testing Against West Nile Virus. The CPE inhibitory assay described in Example 1 was used with the following modifications. Serial dilutions of test compounds were added to lightly confluent Vero cells in 96-well microplates, after which 5× CCID₅₀ of WNV were added to the cells. Uninfected cells, infected cells with no drug and uninfected drug-treated cells were used as controls. 6-azauridine (6-aza-U) was used as the control drug. Duplicates of toxicity controls at each drug concentration and triplicates of test samples were performed. After 6 days post-virus exposure, cells were visually scored for CPE. The EC₅₀ and CC₅₀ were calculated by regression analysis using the means of the CPE ratings at each concentration of the compound.

The results obtained from in vitro evaluation of ZX-2401 and control drug 6-aza-U against two strains of WNV are compiled in Table 1. ZX-2401 showed an excellent activity against both strains of WNV with minimal cytotoxicity to the host cells. The antiviral activity of ZX-2401 was comparable to the control drug. Ribavirin was not used as positive control in WNV experiments because of its poor activity against this virus in tissue culture (Morrey et al., 2002).

TABLE 1
Effect of ZX-2401 on West Nile Virus using neural red assay

| | ATCC VR-82 | | | New York Isolate | | |
|---|---|---|---|---|---|---|
| Compound | EC₅₀ | CC₅₀ | | EC₅₀ | CC₅₀ | |
| (Assay Method) | (μg/ml) | (μg/ml | SI | (μg/ml | (μg/ml | SI |
| ZX-2401 (Visual) | <4.6 | >100 | >21 | 9.5 | >100 | >10.5 |
| ZX-2401 (NR*) | 3.0 | >100 | >33.3 | 8 | >100 | >12.5 |
| 6-aza-U (Visual) | 22 | 85 | 3.8 | 0.9 | 85 | >94.5 |
| 6-aza-U (NR*) | 5 | >100 | >20 | 1.6 | >100 | >62.5 |

*NR = Neutral red

To delineate the actual antiviral effects of test compounds from the potential cytotoxic effects of the compounds, VYR assay was performed as described in Example 1. The results obtained from viral yield study are summarized in Table 2. ZX-2401 reduced virus production when measured at day 2-post initiation (EC₉₀=3.3). The EC₉₀ increased 12-fold (EC₉₀=40) when measured at day 6. This effect of losing antiviral activity at later days into the experiment is similar, but not identical, to that observed with 6-aza-U (Table 2).

TABLE 2
Effect of ZX-2401 or 6-azaurindine on virus yield reduction (VYR)
assay at various time points after initiation of experiment

| | | |
|---|---|---|
| Days after experiment | Virus yield reduction | Inhibition of CPE |
| initiation | assay EC₉₀ | assay EC₅₀ |

ZX-2401

| | | |
|---|---|---|
| Day 2 | 3.3 | Not Determined |
| Day 6 | 40 | 15 |

6-azauridine

| | | |
|---|---|---|
| Day 2 | 1.6 | Not Determined |
| Day 6 | 431 | 3.1 |

Effects of MOI variation on antiviral activity. To investigate the effect of variation in the MOI on the anti-viral activity of ZX-2401, the assays were performed using various MOIs of WNV. The data compiled in Table 3 shows that MOI covering 1.5 log₁₀ did not affect the antiviral activity of ZX-2401. This observation is a favorable indicator for an antiviral compound because it suggests that the compound can be utilized over a wide range of viral burden.

TABLE 3
The effects of MOI on the inhibition of West Nile virus New York
strain in Vero cells by ZX-2401

| | MOI | | | |
|---|---|---|---|---|
| | 1.6 × 10⁻³ | 5 × 10⁻⁴ | 1.6 × 10⁻⁴ | 5 × 10⁻⁵ (5 CCID₅₀^{a}) |
| | EC₅₀ | EC₅₀ | EC₅₀ | EC₅₀ |
| Compound | (μg/ml) | (μg/ml) | (μg/ml) | (μg/ml) |
| ZX-2401 | 20 | 15 | 12 | 15 |

^{a}MOI used in normal antiviral test, 5-50% cell culture infectious doses.

Antiviral Testing Against Hepatitis C Virus in Replicon Assay System. Evaluation of ZX-2401 against HCV was conducted using HCV Replicon assay. The experiment was carried out at Apath LLC (St. Louis, Mo.) according the Apath HCV replicon assay protocol. At the same time, the cytotoxicity of the compound was also determined by measuring the effect on GAPDH mRNA.

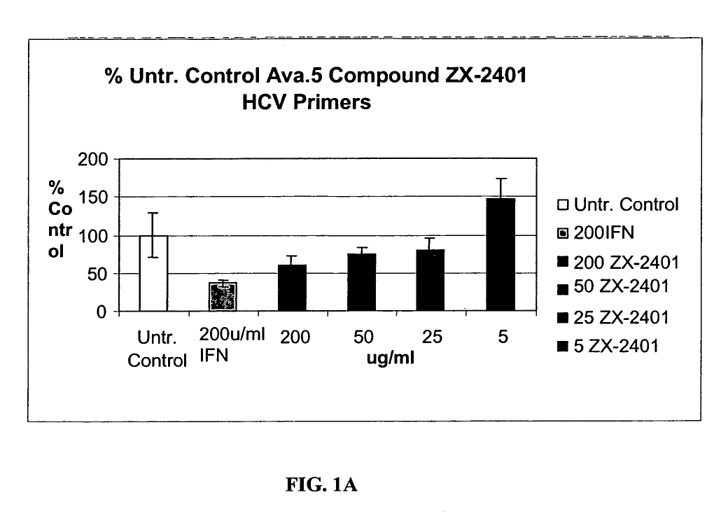

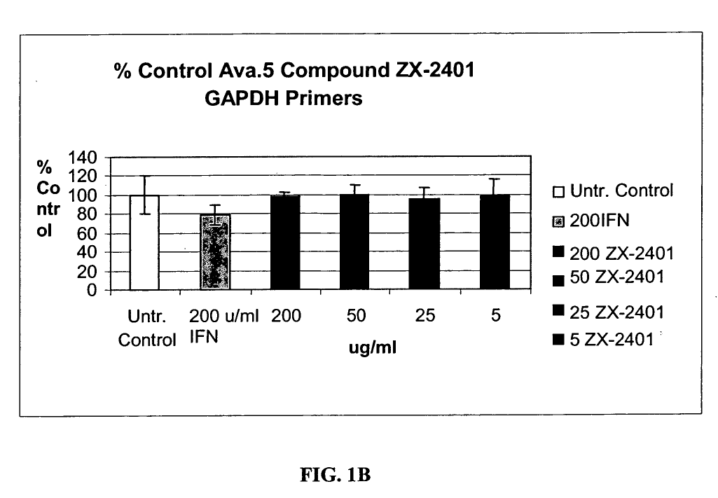

The results of two separate experiments are given in the FIGS. 1A-B. Compound ZX-2401 had a dose-responsive anti-HCV replicon effect while exhibiting no toxicity as measured by GAPDH mRNA levels.

Antiviral Testing Against Other Flaviviruses. On the basis of the antiviral activity observed with WNV and HCV, further experiments were carried out to investigate antiviral spectrum of this compound against other viruses the Flaviviridae family.

Antiviral testing against yellow fever virus. ZX-2401 was tested against YFV 17D strain using a CPE assay system. A known positive control drug (i.e., ribavirin) was evaluated in parallel with ZX-2401 in each test. After appropriate time post-virus exposure, the plates were scored visually, after which neutral red was added to the medium. The EC₅₀ values obtained are presented in Table 4.

TABLE 4
In vitro Effect of ZX-2401 and Ribavirin on YFV, BVDV, BV and DV

| | ZX-2401 | | | Ribavirin | | |
|---|---|---|---|---|---|---|
| | EC₅₀ | CC₅₀ | | EC₅₀ | CC₅₀ | |
| Viruses | (μg/ml) | (μg/ml | SI | (μg/ml | (μg/ml | SI |
| YFV | 10 | >100 | >10 | 32 | >100 | >3.125 |
| BVDV | 0.6 | >100 | >166.6 | 5 | >100 | >20 |
| BV | 5 | >100 | >20 | 60 | >100 | >1.67 |
| DV | 10 | >100 | >10 | >80 | >100 | ND |

ND = Note Determined.

In this study, ZX-2401 inhibited YFV in cell culture with minimum cytotoxicity. ZX-2401 antiviral activity against YFV was up to 3-fold better than ribavirin.

Antiviral testing against dengue virus. Evaluation of ZX-2401 against DV was conducted using CPE assay system described above. As shown in Table 4, ZX-2401 showed excellent activity against DV production in culture. In this experiment, ZX-2401 showed a very superior activity to ribavirin with minimum cellular toxicity. The EC₅₀ values were 10 and >80 μg/ml for ZX-2401 and ribavirin, respectively. In addition, ZX-2401 completely inhibited DV production in cell culture at concentration of 32 μg/ml.

Antiviral testing against bovine viral diarrhea virus. Evaluation of ZX-2401 in a pestivirus was conducted using CPE assay against BVDV. The results shown in Table 4 demonstrated that ZX-2401 inhibited BVDV in a dose-dependent fashion, and in this experiment ZX-2401 was almost 10-fold more active than ribavirin.

Antiviral testing against banzi virus. Evaluation of ZX-2401 in a pestivirus was conducted using CPE assay against BV in Vero cells. The results of this experiment show that ZX-2401 was 12-fold more active than ribavirin (Table 4).

Combination Experiments using ZX-2401 and IFN. The purpose of this study was to investigate the effects of ZX-2401 and IFN in combination using YFV in cell culture. The data obtained from this study is tabulated in Table 5. By itself, ZX-2401 completely reduced viral CPE at 320 and 100 μg/ml, with minimal CPE present at 32 μg/ml. The IFN by itself reduced CPE by 100% at the 100 units/ml dose. There was a dose-responsive effect on CPE reduction between 32 and 3.2 units of IFN. Combinations of ZX-2401 and IFN reduced viral CPE by 100% at all combinations tested. Toxicity of the compounds alone or in combination was assessed by visual inspection of treated uninfected cultures. No toxicity was evident at any combination or when the compounds were used alone.

TABLE 5
Effect of combination ZX-2401 and interferon alpha B/D
on a yellow fever virus infection in Vero cells,
determined by virus titer reduction assay.

| | Virus Titer | | | | |
|---|---|---|---|---|---|
| | (Log₁₀ CCID₅₀/0.1 ml) | | | | |
| ZX-2401 | Interferon (units/ml) | | | | |
| μg/ml | 100 | 32 | 10 | 3.2 | 0 |
| 320 | 0* | 1.7* | 2.0* | 1.7* | 2.3 |
| 100 | 1.3* | 4.0 | 4.7 | 4.3 | 3.7 |
| 32 | 4.7* | 6.3 | 6.3 | 5.3* | 5.7 |
| 0 | 6.0 | 6.5 | 6.5 | 6.3 | 6.5 |

*Indicates improved results compared to those using ZX-2401 or IFN alone at the same dosages.

Even though CPE was inhibited by 100%, there was still virus present in the cultures (Table 5). ZX-2401 by itself produced a dose-responsive inhibition of virus titer, with the highest dose being the most active. IFN was minimally active in suppressing virus titer, even though CPE was inhibited 100% at 100 and 32 units/ml. The combination of IFN at 100 units/ml plus 320, 100, and 32 μg/ml of ZX-2401 reduced virus titer below that which was achieved by ZX-2401 alone.

Though the virus yield reduction assay is subject to variation, as is evident in the data, the results appear clear here that it was only at the highest IFN dose that it combined with ZX-2401 to reduce virus titer. It is interesting that in this cell culture system, viral CPE reduction by itself (100% reduction) was not indicative of dramatic virus titer reductions under many of the conditions shown here.

ZX-2401 alone reduced YFV titer in a dose-dependent manner. IFN α B/D alone may have had a weak effect at 100 units/ml. The combination of ZX-2401 (at 320, 100, or 32 μg/ml) and IFN at 100/units/ml reduced virus titers below that of ZX-2401 alone. No other drug combinations appeared to reduce virus titer below that achieved by ZX-2401 alone. Furthermore when evaluated the combination experiment data in Table 5 using the Combination Index method described by Schinazi et al. (1982) a synergistic antiviral effect was indicated.

In this Example the inventor demonstrated that compound ZX-2401 was capable of inhibiting the production in culture of at least five members of the Flaviviridae family with minimum cytotoxicity. The activity of ZX-2401 appears to be comparable to or better than the control drugs in these studies. Like ribavirin (Poynard et al., 1998), ZX-2401 may be administered in combination with other therapeutic compounds, such as IFN, as a therapy for flavivirus infection. The fact that synergy was observed when ZX-2401 and IFN were used in combination provides a significant indication that this combination of therapies may be particularly beneficial.

It is noted that ZX-2401 did not lose as much antiviral activity as 6-aza-U at 6 days (Table 2). Several explanations for these findings are possible based on results using 6-aza-U. One explanation is that the drugs were labile over time of incubation on the cells, so that with increasing time, they lost efficacy as reflected in the increasing virus titers. Moreover, the CPE might be a delayed response to the initial virus reduction so that CPE-inhibitory effects were not observed until days 4 or 6. This explanation of unstable drug was probably not the cause, because addition of fresh 6-aza-U every 2 days did not improve the VYR at day 6 as compared to no addition of fresh compound. Another explanation is that the drugs acted as metabolic modifiers to slow the replication of the virus and consequently the delayed CPE. Over time, however, the virus titers in the 6-aza-U-treated cells reached the same virus levels as the untreated cells. This is consistent with the observation that 100% CPE was observed 8 days post-virus initiation in cells treated with any concentration of 6-aza-U. A third possible explanation is that minor populations of virus, not responsive to drug treatment, replicated eventually to high levels to overtake the drug-sensitive variants. It is also recognized that explanations for ZX-2401 may or may not be the same as those for 6-aza-U. Nonetheless, it is important to note that ZX-2401 was approximately 10-fold more active than the control drug in this assay.

The mechanism for the beneficial effect of ribavirin remains unclear given that ribavirin appears not to eradicate viral replication in HCV patients. To date, several mechanisms of action (MOA) have been proposed for ribavirin. These include: inhibition of inosine monophosphate dehydrogenase (IMPDH) (Markland et al., 2000), inhibition of proinflammatory mediators induced by viral infection (Ning et al., 1998), and inducement of lethal mutagenesis after incorporation during viral RNA synthesis, which leads to loss in total viral genomic RNA (Crotty et al., 2001). The MOA of ZX-2401 is currently unknown; however, based on of the fact that it is also a nucleoside analog with broad-spectrum antiviral activity, it is conceivable that it would exhibit some but perhaps not all MOA that have been proposed for ribavirin. However, unlike ribavirin, ZX-2401 has strong antiviral activity against WNV, implying a different or additional mode of action. This characteristic, coupled with lack of toxicity to the host cells in tissue culture, suggests that it might lack some of the undesirable effects usually associated with ribavirin. Further experiments are needed to elucidate the difference in mechanism(s) of action between ZX-2401 and ribavirin.

The data described herein suggest that ZX-2401 is a broad-spectrum inhibitor of the RNA viruses. The fact that ZX-2401 is less toxic and more active than ribavirin, a chemically-related compound which is widely used to treat these viruses, strongly argues for the use of ZX-2401 to treat infections caused by the viruses in the Flaviviridae family as a monotherapy or in combination with other therapies such as IFN.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,246,408
U.S. Pat. No. 5,641,515
U.S. Pat. No. 5,580,579
U.S. Pat. No. 5,792,451
U.S. Pat. No. 5,629,001
U.S. Pat. No. 6,753,514
U.S. Pat. No. 6,613,308
U.S. Pat. No. 5,466,468
U.S. Pat. No. 5,543,158
U.S. Pat. No. 5,641,515
U.S. Pat. No. 5,399,363
U.S. Pat. No. 5,466,468
U.S. Pat. No. 5,756,353
U.S. Pat. No. 5,804,212
U.S. Pat. No. 5,725,871
U.S. Pat. No. 5,780,045
PCT Appln. WO 01/17518
A1 et al., Virus Res., 53:141-149, 1998.
Alt et al., Arch. Virol., 142:589-599, 1997.
Alter, J. Am. Soc. Hematol., 85:681-1695, 1995.
Alter et al., N. Engl. J. Med., 327:899-1905, 1992.
Barrera et al., Hepatology, 21:639-644, 1995.
Behrens et al., EMBO J., 15:12-22, 1996.
Cai et al., In: Symposium on Emerging Therapies for Chronic Viral Hepatitis, Session I(5), 1998.
Chambers et al., Annu. Rev. Microbiol., 44:649-688, 1990.
Clarysse et al., In: Congress of the American Association for the Study of Liver Diseases and the International Association for the Study of the Liver, 1998.
Crotty et al., PNAS, 98:6895-6900, 2001.
Davidson et al., J. Infect., 23:241-249, 1991.
Davis, Semin. Liver Dis., 19(Suppl. 1):49-55, 1999.
Davis et al., N. Engl. J. Med., 339:1493-1499, 1998.
De Clercq et al., Antimicrob. Agents Chemother., 35:679-684, 1991.
Edwards et al., Br. Vet. J., 151:181-187, 1995.
Enserink, Science, 286:1450-1451, 1999.
George et al., Bull WHO, 62:879-882, 1984.
Gubler, Clin. Microbiol. Rev., 11:480-496, 1998.
Han et al., J. Infect. Dis., 179:230-233, 1999.
Heinz and Mandl, APMIS, 101:735-745, 1993.
Huggins, Rev. Infect. Dis., 11(Suppl. 4):S750-S761, 1989.
Hwang et al., Crit. Rev. Ther. Drug Carrier Syst., 15(3):243-284.1998.
Ingallinella et al., Biochemistry, 37:8906-8914, 1998.
Kakiuchi et al., FEBS Lett., 421:217-220, 1998.
Kalita and Misra, Clin. Neurophysiol., 106:238-243, 1998.
Karayiannis et al., Clin. Diagn. Virol., 10: 103-111, 1998.
Kim and Caron, Int. Antiviral News, 6:26-28, 1998.
Kim et al., J. Med. Chem., 21:883-889, 1978.
Kramer et al., Am. J. Trop. Med. Hyg., 57:222-229,1997.
Kuno et al., J. Virol., 72:73-83, 1998.
Kurane et al., Arch. Virol. Suppl., 9:59-64, 1994.
Laevens et al., Vet. Q., 20:46-49, 1998a.
Laevens et al., Vet. Q., 20:41-45, 1998b.
Lau et al., C. Hepatology, 28:1121-1127, 1998.
Lavanchy et al., J. Viral Hepat., 6:35-47, 1999.
Lieber and Kay, J. Virol., 70:3153-3158, 1996.
Lieber et al., J. Virol., 70:8782-8791, 1996.
Linnen et al., Science, 271:505-508, 1996.
Lohman et al., Science, 285:110-113, 1999.
Lohmann et al., J. Virol., 71:8416-8428, 1997.
Lu and Wimmer, Proc. Natl. Acad. Sci. USA, 93:1412-1417, 1996.
Mackenzie and Broom, Vet. Microbiol., 46:79-90, 1995.
Mathiowitz et al., Nature, 386(6623):410-414, 1997.
Markland et al., Agents Chemother., 44:859-866, 2000.
Martinot Peignoux et al., J. Hepatol., 29:214-223, 1998.
Meehan et al., J. Comp. Pathol., 119:277-292, 1998.
Misra et al., J. Neurol. Sci., 161:143-147, 1998.
Monath, Proc. Natl. Acad. Sci. USA, 91:2395-2400, 1994.
Monath, Rev. Infect. Dis., 9:165-175, 1987.
Monath and Heinz, In: Flaviviruses,. Fields et al. (Eds), 3rd Ed. 1:961-1034, Lippincott-Raven Publishers, Philadelphia, 1996.
Morrey et al., Antiviral Research, 55:107-116, 2002.
National Institutes of Health, Hepatology, 26, 2S-10S, 1997.
Ning et al., J. Immuno., 160:3487-3493, 1998.
Ohkawa et al., J. Hepatol., 27:78-84, 1997.
Player et al., Proc. Natl. Acad. Sci. USA, 95:8874-8879, 1998.
Poynard et al., Lancet., 352:1426-1432, 1998.
Prince etal., J. Infect. Dis., 167:1296-1301, 1993.
Ramirez et al., Infect. Dis. Clin. North Am., 8:107-128, 1994.
Rappole et al., Emerg. Infect. Dis., 6:319-328, 2000.
Reed and Muench, Am. J. Hyg., 27:493-498, 1938.
Remington's Pharmaceutical Sciences, 18th Ed. Mack Printing Company, 1990. Rice, In: Flaviviridae: the viruses and their replication, Fields et al. (Eds.), Fields Virology, 3rd Ed., 1:931-960, Lippincott-Raven Publishers, Philadelphia, 1996.
Schinazi et al., Antimicrob. Agents Chemother., 22:499-507, 1982.
Smee et al., Antiviral Res., 10:253-262, 1988.
Smee et al., Antiviral Res., 18:151-162, 1992.
Steinkuhler et al., Biochemistry, 37:8899-8905, 1998.
Sudo et al., Antiviral Res., 32:9-18, 1996.
Sudo et al., Biochem. Biophys. Res. Commun., 238:643-647, 1997.
Tan et al., Virology, 216:317-325, 1996.
Takenaga et al., J. Control Release, 52(1-2):81-87 1998
Terpstra and Wensvoort, Vet. Q., 19:97-101, 1997.
Thomas et al., Medine (Baltimore), 74, 212-220, 1995.
Tsai et al., Lancet., 352:767-771, 1998.
Van Campen et al., J. Wildl. Dis., 33:567-573, 1997.
Wakita et al., J. Med. Virol., 57:217-222, 1999.
Welch et al., Gene Ther., 3:994-1001, 1996.
Westaway, Adv. Virus Res., 33:45-90, 1987.
World Health Organization, In: Monograph on dengue/dengue hemorrhagic fever, Regional publication, SEARO No. 22. World Health Organization, Geneva, Switzerland, 1993.
World Health Organization, Dengue bulletin. Bull. W. H. 0. 21:1-15, 1997.
Yamada et al., Br. J. Haematol., 102:616-621, 1998.
Yamashita et al., J. Biol. Chem., 273:15479-15486, 1998.
Yuan et al., Biochem. Biophys. Res. Commun., 232:231-235, 1997.
Zhao et al., J. Virol., 73:1546-1554, 1999.
Zhong et al., J. Virol., 72:9365-9369, 1998.

What is claimed is:

1. A method for treating a Flaviviridae infection comprising administering to a subject a compound having the structure: 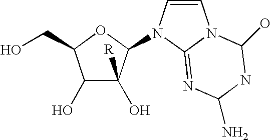
wherein R is chosen from the group consisting of hydrogen, halogen, alkyl, alkoxy, SH, and NH2.

2. The method of claim 1, wherein R is chosen from the group consisting of F, Cl, I, Br, SH, NH₂, CH₃, and —OCH₃.

3. The method of claim 1, wherein R is hydrogen.

4. The method of claim 1, wherein the Flaviviridae infection comprises a Hepacivirus infection.

5. The method of claim 4, wherein the Hepacivirus infection comprises infection with a hepatitis C virus.

6. The method of claim 1, wherein the Flaviviridae infection comprises a Flavivirus infection.

7. The method of claim 6, wherein said Flavivirus infection comprises infection with a yellow fever virus, a dengue virus, a tick-borne encephalitis virus, a St. Louis encephalitis virus, a Japanese encephalitis virus, a Murray Valley encephalitis virus, a Banzi virus, or a West Nile virus.

8. The method of claim 6, wherein said Flavivirus infection comprises infection with a yellow fever virus, a dengue virus, or a West Nile virus.

9. The method of claim 1, wherein the Flaviviridae infection comprises a Pestivirus infection.

10. The method of claim 9, wherein said Pestivirus infection comprises infection with a bovine viral diarrhea virus, a classical swine fever or hog cholera virus, or a border disease virus.

11. The method of claim 1, wherein the subject is a mammal.

12. The method of claim 11, wherein said mammal is a human.

13. The method of claim 11, wherein said mammal is a cow, dog, sheep, pig, cat, horse, mouse, or rat.

14. The method of claim 1, wherein said compound is administered to said subject intranasally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation, injection, infusion, continuous infusion, localized perfusion bathing target cells directly, via a catheter, via a lavage, in cremes, in lipid compositions, or by any combination of the forgoing.

15. The method of claim 14, wherein said compound is administered in a pharmaceutically acceptable carrier, diluent or vehicle.

16. The method of claim 1, further comprising administering to said subject a second anti-viral composition.

17. The method of claim 16, wherein said second anti-viral composition is interferon.

18. The method of claim 17, wherein said interferon is α-2b interferon, α-2a interferon, consensus interferon, or α-1n interferon.

19. The method of claim 16, wherein said second anti-viral composition is ribavirin, ribavirin-2′,3′,5′-triacetate, polyriboinosinic-polyribocytidylic acid, 10-carboxymethyl-9-acridanone, mycophenolic acid, or EICAR.

20. The method of claim 16, wherein said second anti-viral composition is tiazofurin, selenazofurin, a polyanion, a bicyclam, pirodavir, polysulfate PAVAS, or a plant lechtin.

21. A compound having the structure: 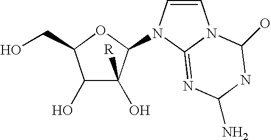
wherein R is chosen from the group consisting halogen, alkyl, alkoxy, SH, and NH₂.

22. The compound of claim 21, wherein R is chosen from the group consisting of F, Cl, I, Br, SH, NH₂, CH₃, and —OCH₃.
